(19) United States
(12) Patent Application Publication
(10) Pub. No.: US 2023/0372129 A1
(43) Pub. Date: Nov. 23, 2023

(54) Prosthetic Suspension Interfaces

(71) Applicants: Aldo Laghi, Pinellas Park, FL (US); Alps South Europe, S.R.O., Plzen (CZ)

(72) Inventors: Aldo Laghi, Pinellas Park, FL (US); Nathaniel Vint, Palm Harbor, FL (US); David, Clearwater, FL (US); Jacob McClelland, Gulfport, FL (US)

(21) Appl. No.: 18/221,522

(22) Filed: Jul. 13, 2023

Related U.S. Application Data
(63) Continuation-in-part of application No. 17/876,764, filed on Jul. 29, 2022.
Application No. 17/876,764 is a continuation-in-part of application No. 17/546,510, filed on Dec. 9, 2021.
Application No. 17/546,510 is a continuation of application No. 16/258,621, filed on Jan. 27, 2019, now Pat. No. 11,224,526.
Application No. 16/258,621 is a continuation-in-part of application No. 16/120,791, filed on Sep. 4, 2018, now Pat. No. 11,179,252.
(60) Provisional application No. 62/554,370, filed on Sep. 5, 2017.

(51) Int. Cl. A61F 2/80; A61F 2/78

(52) U.S. Cl. CPC A61F 2/80; A61F 2/7812; A61F 2002/5007

(57) ABSTRACT

A suspension interface having a fabric layer with an open proximal end and a closed distal end. The fabric layer can be knit or woven and is preferably made from a stretchable, breathable material such as nylon. At a pre-determined point along the length of the fabric layer, an external sealing element is affixed using conventional gluing means creating a bond layer. The suspension interface has an internal grip layer or external grip layer that is preferably silicone but may also be a thermoplastic material such as urethane. The internal or external grip layer can be as thick as one inch in thickness and as thin as 1/64 of an inch but is preferably about ⅛ of an inch.

### CROSS-REFERENCE TO RELATED APPLICATIONS

This application is a continuation-in-part of application Ser. No. 17/876,764, filed Jul. 29, 2022, entitled “Prosthetic Liner with External Elastomeric Seal,” which is a continuation-in-part of application Ser. No. 17/546,510, filed Dec. 9, 2021, entitled “Prosthetic Liner with External Fabric Seal” which is a continuation of application Ser. No. 16/258,621, filed Jan. 27, 2019, entitled “Prosthetic Liner with External Fabric Seal,” which is a continuation-in-part Ser. No. 16/120,791, filed Sep. 4, 2018, entitled “Prosthetic Liner with External Fabric Seal,” which claims priority to U.S. Provisional Application No. 62/554,370, filed Sep. 5, 2017, the contents of which are hereby incorporated by reference herein.

### BACKGROUND OF THE INVENTION

### Field of the Invention

This invention relates to prosthetic socks having an interface for use in a prosthetic assembly. More particularly, this invention relates to knit or woven suspension socks having at least one sealing band internal or external in addition to an externally fabricated sealing ring.

### Description of the Background Art

It has long been appreciated that differential air pressure may be utilized to retain a prosthetic limb on a patient's limb or stump. Gravitational and other forces tend to cause separation between the prosthetic limb and the patient's residual extremity during use. This happens, for example, during the swing phase of gait, when a prosthetic leg is additionally subjected to centrifugal forces. Patients have routinely worn a variety of belts, straps, cuffs and harnesses to retain their prosthetic limbs against separation from the residual limb. In addition, a variety of socket configurations have been utilized for the same purpose. However, none of these can provide the reliable, strong retentive force which is provided by differential air pressure in accordance with the present invention.

Most amputees wear woven “stump socks” as an interface between their skin and the prosthetic socket for increased comfort, perspiration absorption, and reduced friction. These socks vary from thin sheaths (about 0.010-0.015 inches thick) to rather thick socks (about 0.125 inches thick) which are used to accommodate substantial discrepancies between stump shapes and volumes in relation to the prosthetic socket. All of these socks, regardless of the material from which they are fabricated (nylon, orlon, cotton, wool, etc.) are porous. As such, air flows freely past the proximal borders of the socket, making it impossible to maintain a vacuum within. The same problem of uncontrolled air leakage can exist when no stump sock is worn.

The primary issue with most prosthetic devices is breathability because of heat retention. When a gel (thermoplastic, silicone, urethane) is in contact with the skin, the body's normal heat generation can causes sweating which can then cause skin irritation or other skin issues. Skin health drastically improves when it is allowed to breathe. In addition, most prosthetic devices are not machine-washable.

Therefore, it is an object of this invention to provide an improvement which overcomes the aforementioned inadequacies of the prior art devices and provides an improvement which is a significant contribution to the advancement of the prosthetic interface art.

Another object of the invention is to provide a suspension interface that is comfortable for a user while still providing suspension.

Another object of the invention is to prevent bleed-through of the material used for the internal or external sealing band through the fabric layer.

Another object of the invention is to provide a suspension interface that includes a sealing band in addition to an external sealing ring for additional support and suspension.

Another object of the invention is to provide a suspension interface that allows for introduction of a pin locking system to provide additional support and suspension.

Another object of the invention is to create a convenient, improved sealing arrangement between an elastomeric liner sleeve and the interior of a prosthetic socket.

Another object of the invention is to provide a suspension interface that is lightweight, breathable, and machine washable.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

The present invention relates generally to a suspension interface having a fabric layer with an open proximal end and a closed distal end. The fabric layer can be knit or woven and is preferably made from a stretchable, breathable material such as polyester or other synthetic fiber. At a pre-determined point along the length of the fabric layer, an external sealing element is affixed using conventional gluing means creating a bond layer as described above in the related embodiments and having the same configuration. The suspension interface has an internal grip layer or external grip layer that is preferably silicone but may also be a thermoplastic material such as a thermoplastic elastomer, urethane, or similar material. The internal or external grip layer can be as thick as one inch in thickness but is preferably about ⅛ of an inch.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 is a cross-sectional view of the thermoplastic elastomeric layer;

FIG. 2 is a cross-sectional view of the preferred embodiment of the suspension sleeve liner;

FIGS. 3-11 are cross-sectional views of suspension sleeve liners corresponding to FIG. 2 wherein alternate embodiments of the raised and recessed portions are shown;

FIGS. 12-13 are cross-sectional views of suspension sleeve liners wherein alternate embodiments of the raised portions are shown;

FIGS. 14-15 are cross-sectional views of suspension sleeve liners wherein alternate embodiments of the raised portions are shown, specifically liners having an elastomeric material impregnated within a fabric exterior layer;

FIG. 16 is a perspective view of an alternative embodiment of the thermoplastic elastomeric liner wherein the sealing element is glued to the fabric exterior;

FIG. 17 is a cross-sectional view of FIG. 16 along line A-A;

FIG. 17-1 is a magnified view of the cross-sectioned portion showing Detail B of the sealing element area of FIG. 16;

FIG. 18 is a perspective view of the thermoplastic elastomeric liner wherein multiple sealing elements are glued onto the fabric exterior;

FIGS. 19-21 are perspective views of the thermoplastic elastomeric liner showing the sealing element in various locations on the liner;

FIGS. 22 and 23 are perspective views of the thermoplastic elastomeric liner prior to the sealing element being glued to the fabric exterior;

FIG. 24 is a perspective view of another alternative embodiment wherein the thermoplastic elastomeric liner includes a lock at the distal end as well as the sealing element;

FIG. 25 is a cross-sectional view of FIG. 24 along line C-C;

FIG. 26 is a front perspective semi-transparent view of an embodiment of the present invention;

FIG. 27 is a cross-sectional view of the liner shown in FIG. 26 along line D-D;

FIG. 28 is a cross-sectional view of an alternative embodiment of the present invention;

FIG. 29 is a cross-sectional view of the liner shown in FIG. 28 along line E-E;

FIG. 30 is a front perspective semi-transparent view of an embodiment of the present invention;

FIG. 31 is a cross-sectional view of the liner shown in FIG. 30 along line F-F;

FIG. 32 is a front perspective view of an embodiment of the present invention;

FIG. 33 is a cross-sectional view of the liner shown in FIG. 32 along line G-G;

FIG. 34 is a front perspective view of an embodiment of the present invention;

FIG. 35 is a cross-sectional view of the liner shown in FIG. 34 along line J-J;

FIG. 36 is a front perspective view of an embodiment of the present invention;

FIG. 37 is a cross-sectional view of the liner shown in FIG. 36 along line K-K.

Similar reference numerals refer to similar parts throughout the several views of the drawings.

### DETAILED DESCRIPTION OF THE PREFERRED EMBODIMENT

The following description is of the best mode presently contemplated for carrying out the invention. This description is not to be taken in a limiting sense, but is made merely for the purpose of describing one or more preferred embodiments of the invention. The scope of the invention should be determined with reference to the claims.

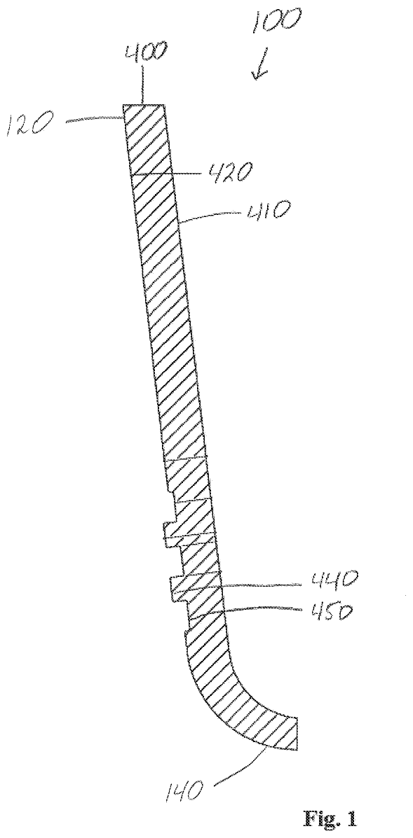

FIG. 1 illustrates a thermoplastic elastomeric layer 400 of the seal liner 100 in which the thermoplastic elastomer layer 400 (also known as TPE) having an inner elastomer surface 410 and outer elastomer surface 420 is formed within a mold having wall thicknesses between 2 and 9 mm. The liner 100 preferably has an open proximal end 120 with a larger circumference and volume than of the closed distal end 140.

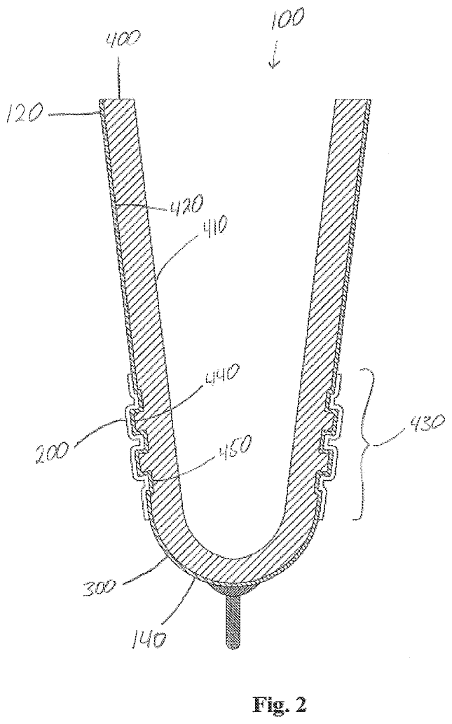

FIG. 2 illustrates a preferred embodiment of the seal liner 100 in which the thermoplastic elastomer layer 400 further comprises at least one sealing region 430 that extends preferably between 3 and 20 mm outward from the interior surface 410 of the thermoplastic elastomeric layer 400 and includes at least one raised portion 440. If more than one raised portion 440 is included within the sealing region 430, the plurality of raised portions 440 will be accompanied by at least one recessed portion 450. Thus, if there are two raised portions 440 then three recessed portions 450 will be distributed as shown in FIG. 1. Alternatively, the sealing region 430 may begin with a raised portion 440 instead of a recessed portion 450. In this instance, the distribution would be as follows: raised portion 440, recessed portion 450, raised portion 440. Thus, the recessed portions 450 do not always have to surround distal and proximal ends of the sealing region 430.

A fabric exterior 300 is synonymous with “reinforcement layer” and is bonded to the outer elastomer surface 420 of the thermoplastic elastomer layer 400 and conforms to the at least one sealing region 430. The fabric exterior 300 advantageously forms a suitable substrate for bonding a seal layer 200. The seal layer 200 conforms to the shape of the fabric exterior 300 and thermoplastic elastomer layer 400. The seal layer 200 is applied in an uncured state to the fabric and with a viscosity such that it can “wet out” the fabric in that area so that once cured it forms an air-tight seal thus isolating the fabric above the seal layer (not shown) from the fabric exterior 300 below the seal layer 200. Thus, this application of the outer sealing layer 200 to the fabric exterior 300 provides an air-impermeable interface between the thermoplastic elastomeric layer 400 and the surrounding exterior area of the liner 100. The outer sealing layer 200 is preferably applied such that it impregnates the reinforcement layer 300 to form a composite.

This outer sealing layer 200 may be comprised of natural rubber, silicone, polyurethane, latex, polysulfide, vinyl, polyisoprene, or a styrene block copolymer gel, although the preferred material is rubber due to their high abrasion resistance and high force to stretch. The force to stretch this layer is determined by the modulus of elasticity of the sealing material multiplied by the cross-sectional area of the sealing material.

The thermoplastic elastomer layer 400 is preferably of a type compatible with long periods of dynamic wearer contact. Such materials are known in the art and may include the following polymers, as well as gels which comprise them: silicones polyurethanes; block copolymers such as styrene block copolymer gels, general non-limiting examples of which may include SEBS-, SEPS-, SEEPS-, SEEBS-, and other type styrene block copolymer gels. Further non-limiting examples of styrene block copolymer gels which may be useful in the liner of the present invention include so called “controlled distribution polymers,” such as, for example, those disclosed in U.S. Pat. No. 7,226,484; United States Patent Application Publication No. 20070238835; and United States Patent Application Publication No. 20050008669, the disclosure of which is hereby incorporated by reference herein. Other potentially useful polymers may include certain so-called “crystalline” polymers, such as, for example, polymers disclosed in U.S. Pat. Nos. 5,952,396; 6,420,475 and 6,148,830, the disclosures of which are hereby incorporated by reference herein. The above list is non-limiting, and in general, the list of acceptable polymers and gels includes those known in the art to be useful for the fabrication of prosthetic liners. The term “gel” is defined to be a polymer mixed with a plasticizer. An example of current liner using such gel is the “EZ Gel”™ liner, available from Alps South, LLC.

The term “sealing region” 430 may include only one raised portion 440 or may include a plurality. If it includes a plurality of raised portions 440, the sealing region 430 will have each raised portion 440 contiguously adjacent to any other raised portion 440. However, if there are a plurality of raised portions 440 collectively near the distal end of the tubular body portion as well as a plurality of raised portions 440 collectively near the proximal end of the tubular body portion, there will be two sealing regions 430 with only continuous lengths among their group. That is, the sealing region 430 in the distal end will have one continuous length of material while the sealing region 430 in the proximal end will have its own continuous length of material. The continuous length of material refers to the elastomeric material used to create the durable and flexible sealing region 430.

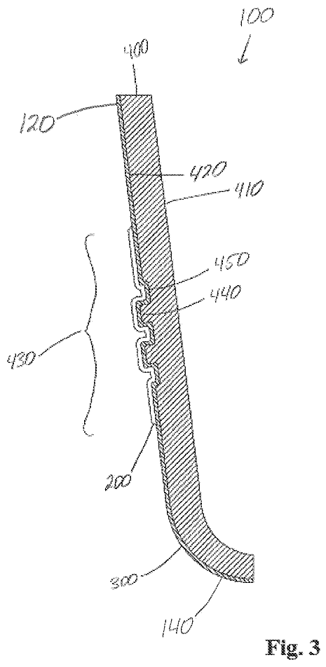

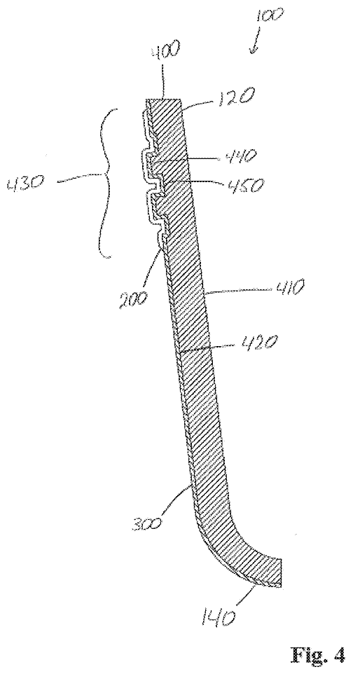

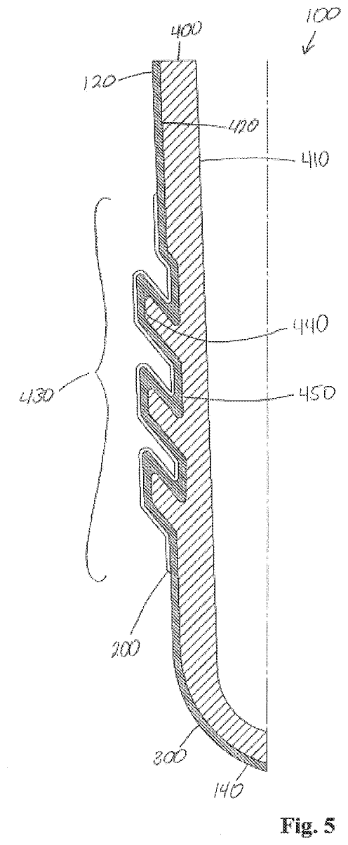

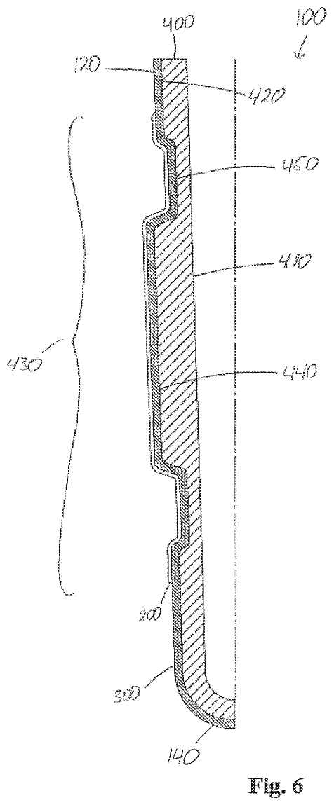

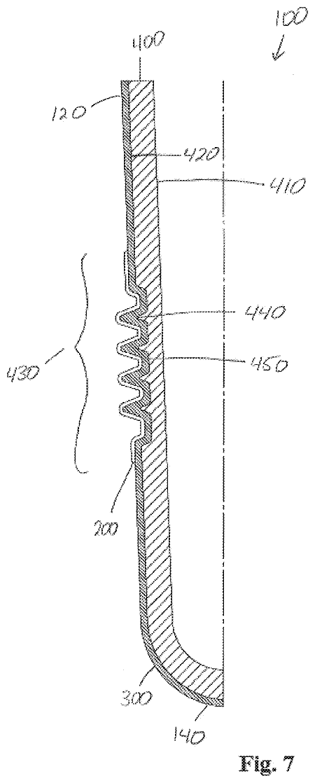

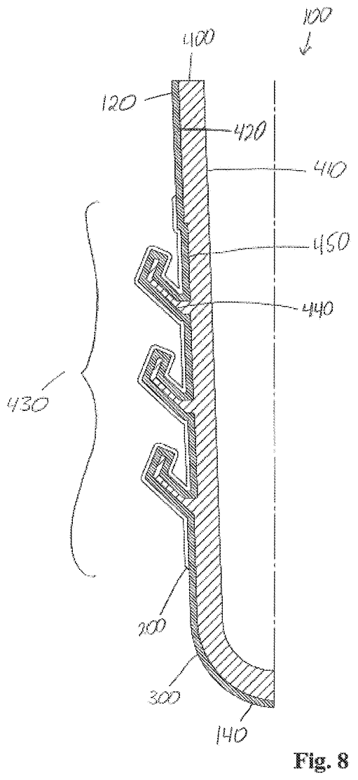

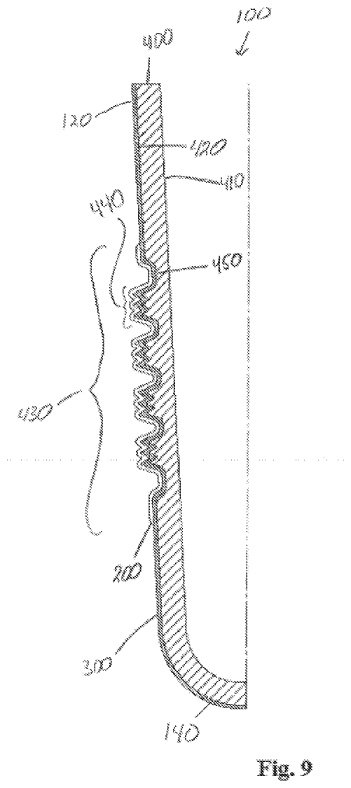

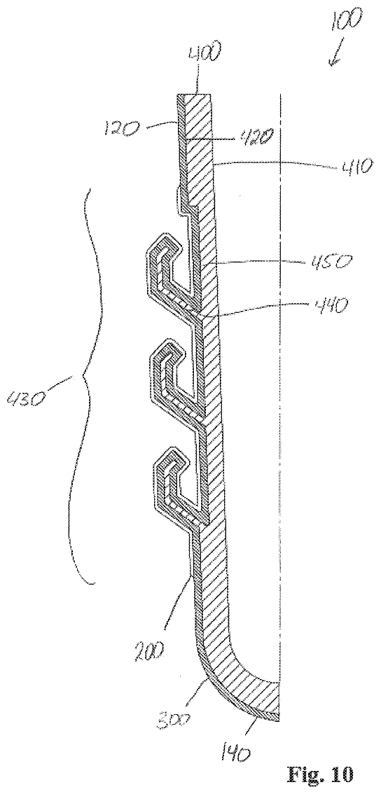

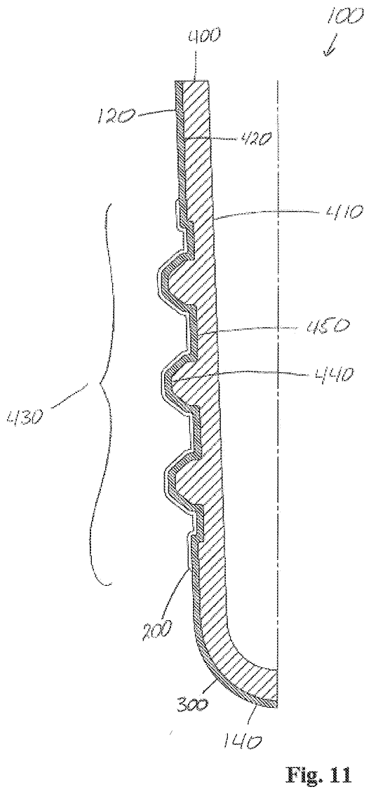

FIGS. 2-11 show that the raised portions 440 within the elastomeric material can have a multitude of different geometries relative to the vertical axis of the invention. For example, the raised portions 440 can have a square/rectangular (FIGS. 2-4, 6), parabolic (FIG. 11), triangular, oval, spear-like, parallelogram-like (FIG. 5), dome-like (FIG. 11), ripples (FIGS. 7 and 9), or protrusions that resemble “gripping” mechanisms or shapes that one skilled in the art would use to “grip” an object to another object. For example, a finger-like projection (FIGS. 8 and 10) that resembles a hook similar to the “hook” portion of a “hook and loop” configured as used in Velcro®. Each raised portion 440 preferably is not co-linear with the fabric exterior layer 300, as can be seen in the relevant figures.

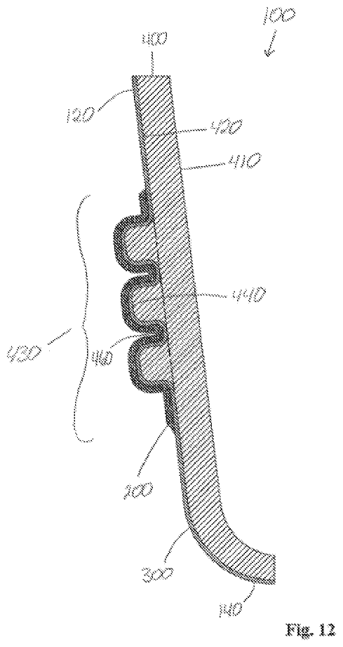

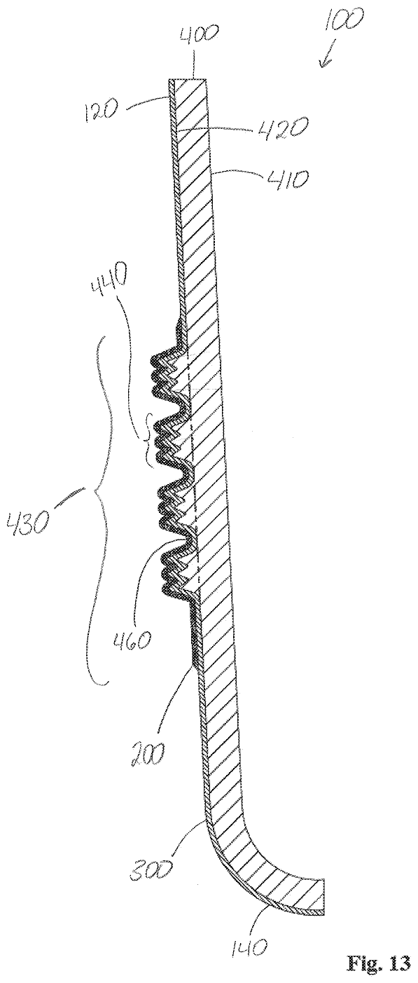

FIGS. 12 and 13 show the seal liner 100 without recessed portions 450 in the elastomeric layer 400. The raised portions 440 protrude from the elastomeric layer 400 without recessed portions 450 located between, among, or near the raised portions 440. This embodiment of the seal liner 100 is useful because of its ease of manufacturing and slimmer profile. Only the reinforcement layer 200 and fabric exterior layer 300 have recessed portions 450 which correspond to the recesses 450 between each raised portion 440.

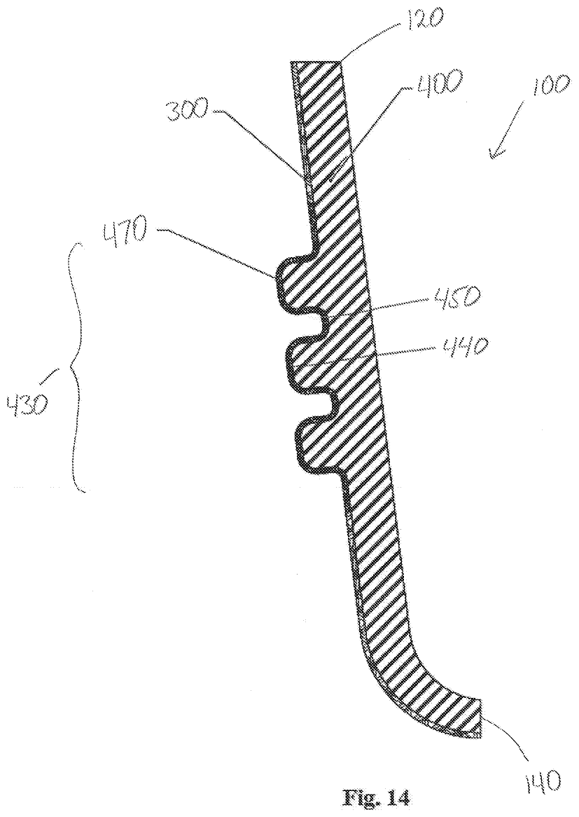

FIG. 14 shows an additional embodiment of the seal liner 100 which includes having elastomeric material impregnated within the fabric exterior 300 along the entire length of the sealing region 430. The elastomeric material penetrates at least partially within the fabric exterior 300 and, preferably, is completely impregnated within the fabric exterior 300.

Furthermore, having the elastomeric material impregnated within the fabric exterior 300 allows the resulting composite to have more durable properties as compared to having the elastomeric material attached as its own layer apart from the fabric exterior 300 (FIGS. 2-13). That is, the fabric exterior 300 in conjunction with the impregnated elastomeric material has similar characteristics as having reinforcing bars (“rebar”) positioned within concrete structures.

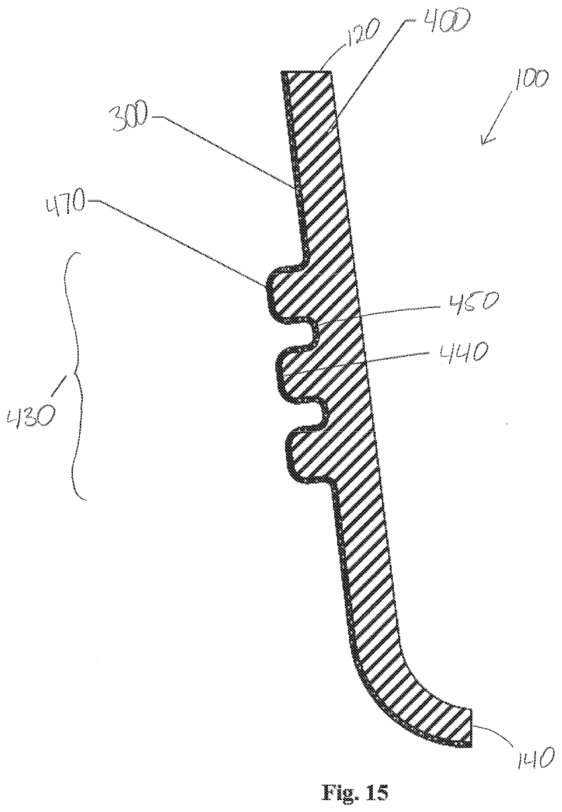

FIG. 15 shows the uses of having an apex surface 470 of the raised portions 440. The elastomeric material is fully impregnated within the fabric exterior 300 only on the apex surface 470 of the raised portions 440, as opposed to FIG. 14, which shows the elastomeric material impregnated into the fabric exterior 300 along the entire length of the sealing region 430.

Furthermore, the apex surface 470 is just that, the highest or furthest point from the recessed portion 450. In the case of the embodiment shown in FIG. 7, for example, the apex surface 470 would be relatively at the “tip” of the triangular-like raised portion 440 and extend down towards the recessed portion 450. In FIG. 8, the apex surface 470 would be the end of the finger-like projection. The apex surface 470 may also be viewed as the surface area of the raised portion 440 that comes into contact with the socket wall.

Preparing the embodiment shown in FIGS. 14 and 15 involves four steps. First, a tube having an inflatable bladder is inserted into the seal liner 100. The inflatable bladder is positioned to be near the sealing region 430. Vinyl tape is then used to wrap the areas of the raised portions 440 to prevent any unwanted silicone from bonding to any other area apart from the apex surface 470 of the raised portions 440. The borders created from the applied vinyl tape will define the edges of the seal area. Compressed air is then used to inflate the bladder, which will stretch the fabric exterior 300 to allow the silicone to penetrate the fabric exterior 300. The tube is then placed on a rotating arm which will rotate the seal liner 100 to allow uniform coating of the apex surface 470 of the raised portions 440, while preventing the silicone from dripping by using the principle of centrifugal force.

Second, a calibrated scale and clean mixing container are used to prepare and mix the required silicone to be applied to the apex surface 470 of the raised portions 440. Although in this method, silicone is used as the sealing material, any equivalent material may be used.

Third, the seal liner 100 is rotated while a brush or other applier is used to apply the prepared silicone to the apex surface 470 of the raised portions 440. The applied silicone is uniform and only introduced to the areas within the sealing region 430 that do not have the vinyl tape applied. No bubbles should be present within the applied silicone. The bladder is then deflated and vinyl tape removed.

Fourth, the seal liner 100 is slowly rotated on the rotating arm while an infrared heat lamp is applied approximately 1 inch from the surface of the silicone. The seal liner 100 is left to cure for approximately 10 to 20 minutes or until the silicone is no longer tacky to the touch. If the silicone coating is still liquid or soft, the seal liner 100 is left rotating to cure longer. Once the silicone is cured enough to remain in place without rotating, the seal liner 100 is moved to a clean location to finish curing overnight at room temperature.

Moreover, the impregnating process of the silicone onto the fabric exterior 300 can be accomplished in a variety of ways, including applying pre-made, semi-cured silicone strips onto the apex surface 470 of the raised portions 440. The process of applying may include painting, spraying, coating, spreading, injecting, forming, or dipping the seal liner 100 into the silicone material without inflating the seal liner 100.

Many stress and abrasion tests were successfully performed to show that the seal liner 100 would retain a vacuum while in use because of its durable sealing region 430. The seal liner 100 was first subjected to a vacuum test to establish a baseline for the level of vacuum achieved within the seal liner 100 and socket (vacuum achieved 23 inches Hg, vacuum maintained 20 inches Hg, and the duration of test was 24 hours). After the baseline was established, the elastomeric material impregnated within the fabric exterior layer 300 was gradually removed by abrasive forces to monitor the resulting vacuum level. The first sanding step removed a small layer of the elastomeric material impregnated within the fabric exterior 300 at the apex surface 470 of the raised portions 440 (vacuum achieved 22.5 inches Hg, vacuum maintained 20 inches Hg, and the duration of test was 24 hours). There was no noticeable loss of vacuum. The second sanding step removed a much thicker layer of elastomeric material impregnated with the fabric exterior 300 and again, no noticeable loss of vacuum was achieved (vacuum achieved 18.5 inches Hg, vacuum maintained 17.5 inches Hg, and the duration of test was 24 hours). The sanding steps continued until progressively, each raised portion 440 had a significant loss of impregnated elastomeric material within the fabric exterior 300 (vacuum achieved 23 inches Hg, vacuum maintained 20 inches Hg, and the duration of test was 24 hours). No noticeable loss of vacuum was achieved. Over the course of the many trials of the sanding steps, the seal liner 100 never lost more than 4.5 inches Hg and on average never lost more than 2 inches Hg.

When worn by an amputee and inserted into a socket for use with a vacuum system, the volume of air within the socket and below the seal layer is evacuated thereby securing the residual limb within the socket. The present invention thereby preferably reduces any potential for a tourniquet effect created by the vacuum process and provides high abrasion resistance.

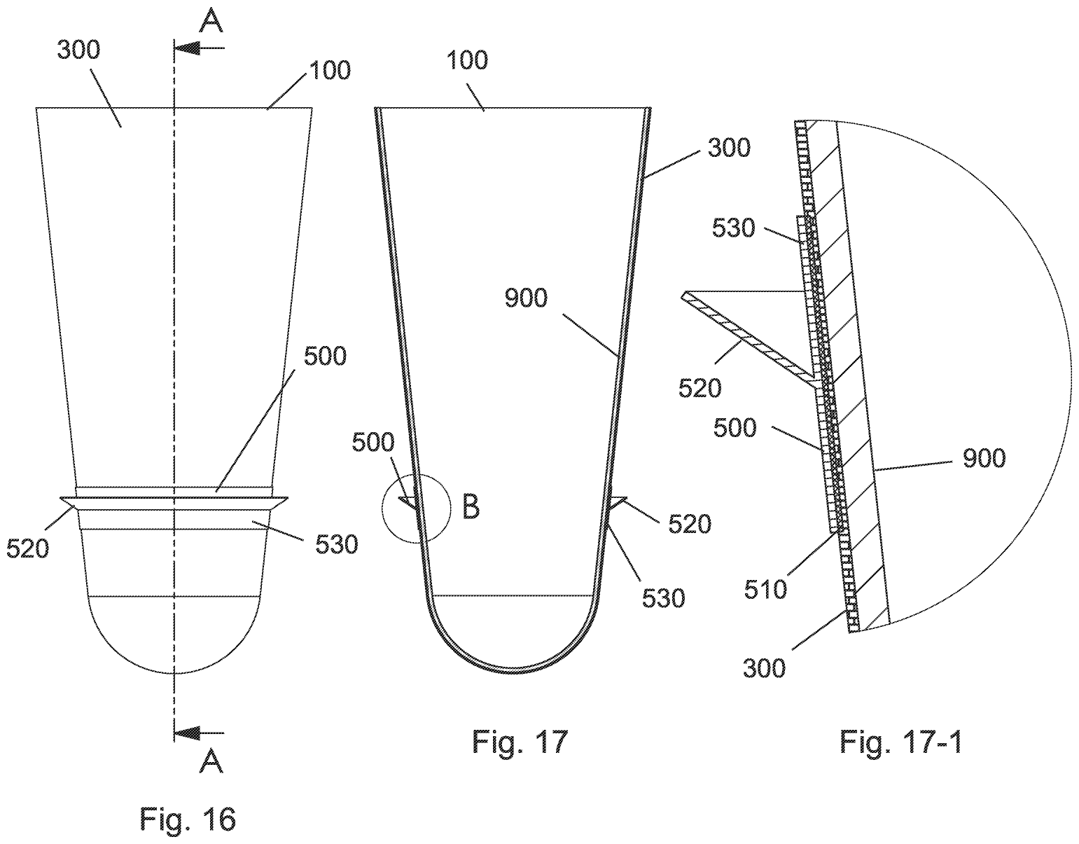

An alternative embodiment is provided for in FIGS. 16-25. Here, as shown in FIGS. 16-17, instead of having the elastomeric seal be integral with the thermoplastic elastomer layer 400 and impregnating the fabric exterior 300, the seal liner 100 will have a sealing element 500, which can be made of either silicone or an elastomeric compound such as urethane, which is non-unitary with either the fabric exterior 300 or a cushioning layer 900, which can be made of either silicone or a thermoplastic elastomer such as SEEPS. This sealing element 500 is preferably made from either silicone or some other thermoplastic elastomer and is bonded to the fabric exterior 300 of the seal liner 100 thereby forming a bond layer 510. Preferably the bond of the bond layer 510 is a type of glue which creates a seal thereby forming a vacuum. The sealing element 500 further comprises an engaging member 520 which extends radially outwardly from the sealing element base 530. The engaging member 520 can be substantially straight, as shown in FIG. 17-1, or may be arcuate, as seen in FIG. 23. A benefit of this embodiment is that it allows for the thermoplastic elastomer layer 400 to be made from a material that is different from the sealing element 500. For example, the sealing element 500 can be made of silicone while the cushioning layer 900 could be made from a SEEPS gel or vice versa.

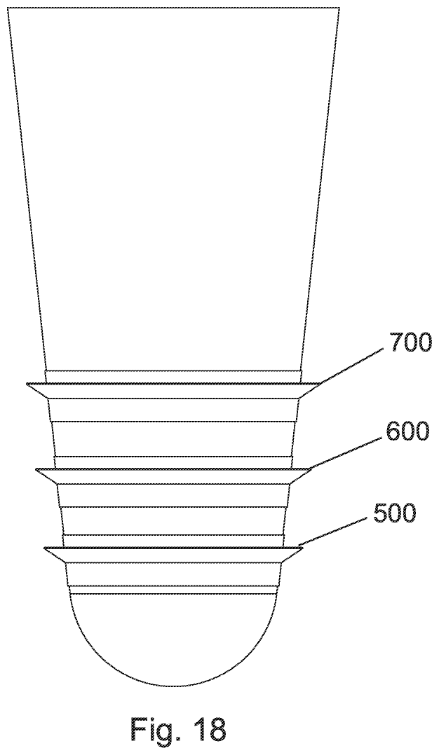

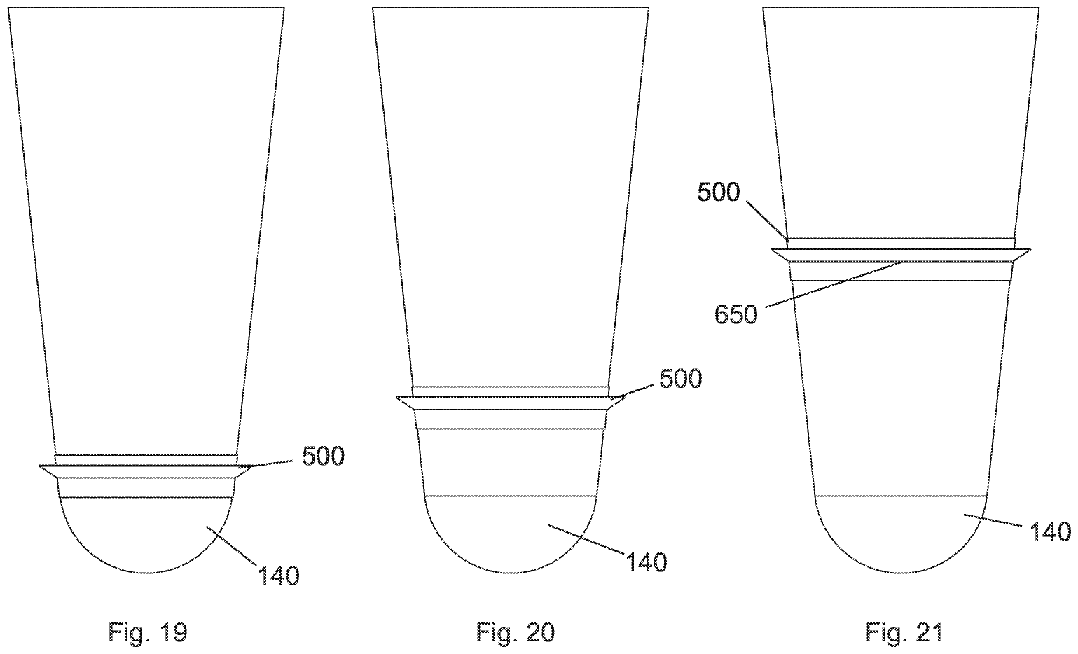

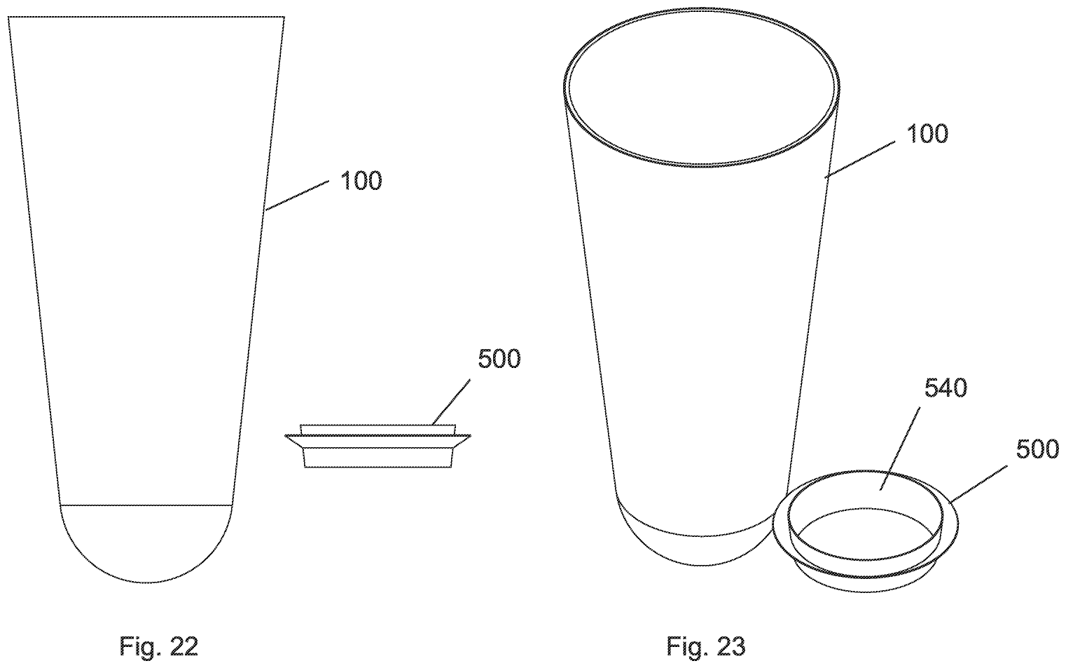

As shown in FIG. 18, the seal liner 100 may have one sealing element 500 but may have up to two additional sealing elements 600, 700 which may be bonded in any position on the seal liner 100 but is preferably located near the closed distal end 140 in order to provide the greatest level of support to the user. Alternatively, as shown in FIGS. 19-21, the sealing element 500 may be placed in different locations on the seal liner 100; at the closed distal end 140 as seen in FIG. 19, about a third of the distance from the closed distal end 140 as seen in FIG. 20, and at a mid-point 650 of the seal liner 100 as seen in FIG. 21. As shown in FIGS. 22 and 23, the sealing element 500 is initially separate from the seal liner 100 and glue is applied only to the interior surface 540 of the sealing element 500.

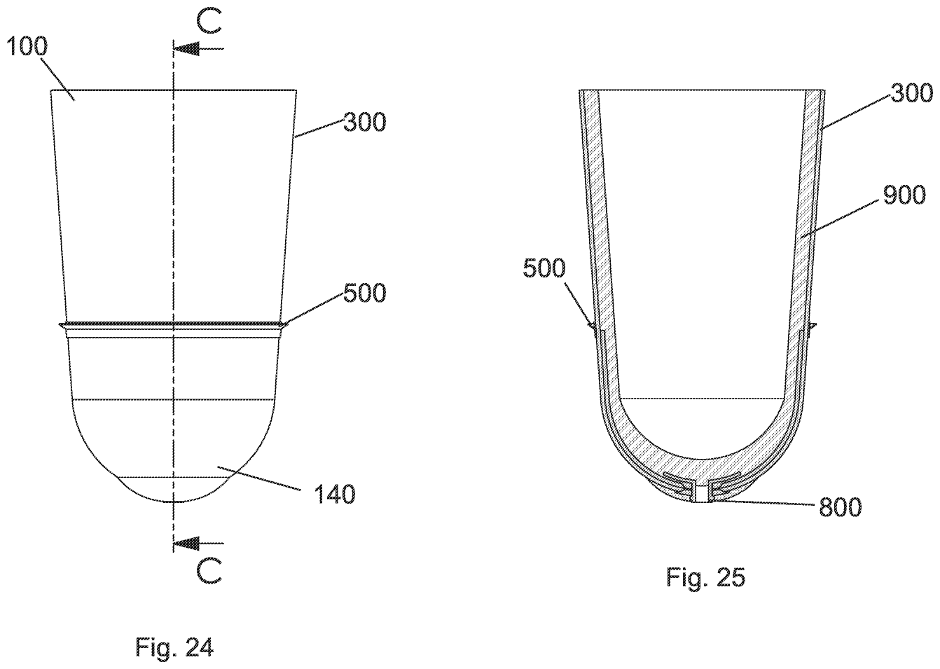

In yet another embodiment, as shown in FIGS. 24 and 25, the sealing element 500 can be used on liners incorporating a distal lock 800 at the closed distal end 140. Such a distal locking system has been previously disclosed in commonly owned U.S. Pat. No. 11,224,527 to Laghi. The benefit of this arrangement is further support for the user while a prosthesis is in use. As in the previous embodiments, the sealing element 500 is glued to the fabric exterior 300.

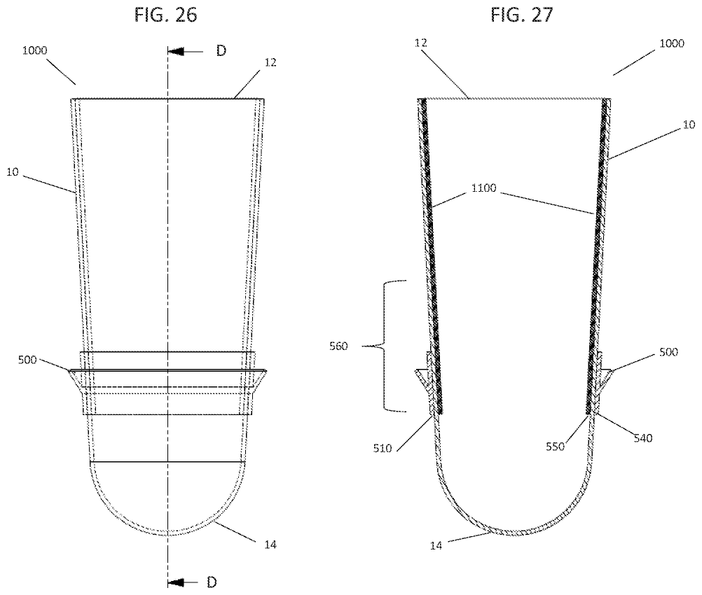

As shown in FIG. 26 through FIG. 32, an alternative embodiment of the present invention incorporates the sealing element 500 of the previous embodiment as part of a suspension interface 1000, preferably as a sock. The suspension interface 1000 generally has a fabric layer 10 with an open proximal end 12 and a closed distal end 14. The fabric layer 10 can be knit or woven and is preferably made from a stretchable, breathable material such as nylon, polyester, or other synthetic fiber. Natural fibers such as cotton, wool, or bamboo may also be used. Furthermore, an elastic yarn made of materials such as Lycra, latex, or rubber can be incorporated into the fabric layer 10 at the closed distal end 14 so that so that the suspension interface 1000 can conform to the shape of the residual stump. It is imperative that the fabric layer 10 be “breathable” which means that it allows the evaporation of sweat formed from activity. At a pre-determined point along the length of the fabric layer 10 based on patient need, generally in an intermediate region 560 defined as about a third of the length of the suspension interface 1000 down from the open proximal end 12 at its uppermost point and about a third of the length of the suspension interface 1000 up from the closed distal end 14, the external sealing element 500 is affixed using conventional gluing means creating a bond layer 510 as described above in the related embodiments and having the same configuration. Internally, the suspension interface 1000 has an internal grip layer 1100 on an inner face 16 of fabric layer 10 that is preferably silicone but may also be a thermoplastic material such as a thermoplastic elastomer comprising styrene blocks, urethane, or a similar material. The internal grip layer 1100 can be as thick as one inch in thickness and as thin as 1/64 of an inch but is preferably about ⅛ of an inch. As shown in FIG. 27, the internal grip layer 1100 can begin at the open proximal end 12 and can extend down to about the location of a distal edge 540 of the sealing element 500 on an interior face 550 of the fabric layer 10.

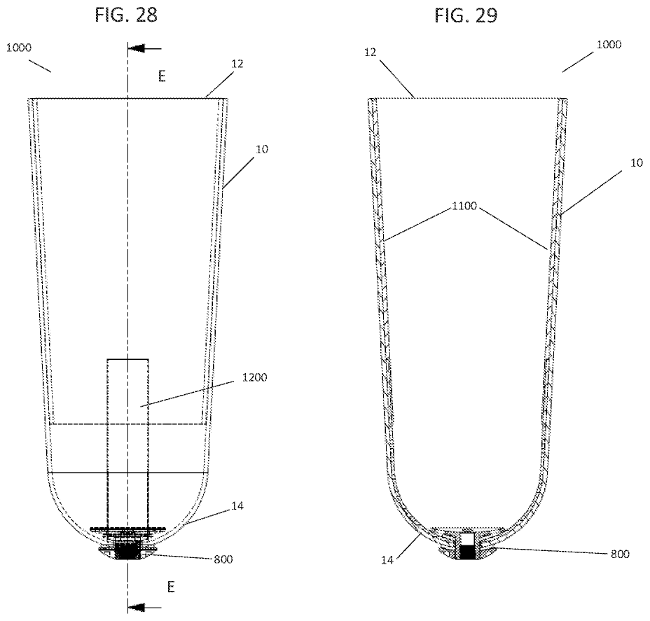

An alternative embodiment of the suspension interface 1000 is shown in FIG. 28 and FIG. 29. In this embodiment, suspension interface 1000 further includes a distal lock 800 in the same configuration as shown in FIGS. 24 and 25. Similar to the embodiment of FIG. 26 and FIG. 27, the suspension interface 1000 includes an internal grip layer 1100 as well as an internal reinforcement strip 1200. This reinforcement strip 1200 is preferably made of a non-elastic material such as silk, fiberglass cloth, carbon fiber, and thermoplastics. The reinforcement strip 1100 may also have an inelastic backing. The reinforcement strip 1200 is placed at the closed distal end 14 of the suspension interface 1000 and extends towards the open proximal end 12 stopping at least one inch from the open proximal end 12.

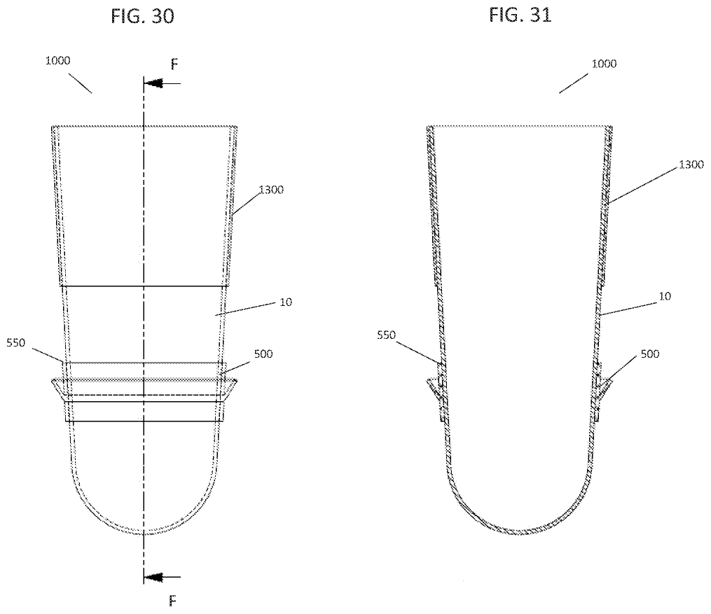

As shown in FIGS. 30 and 31, the suspension interface 1000 can have an external grip layer 1300 located on an exterior face 18 of fabric layer 10. Much like the internal grip layer 1100, the external grip layer 1300 extends from the open proximal end 12. The external grip layer 1300, by virtue of being on top of the fabric layer 10 like the sealing element 500, has a length that is at a maximum to a proximal edge 550 of the sealing element 500. Preferably, the embodiments of FIGS. 26-31 do not bleed through the fabric layer 10.

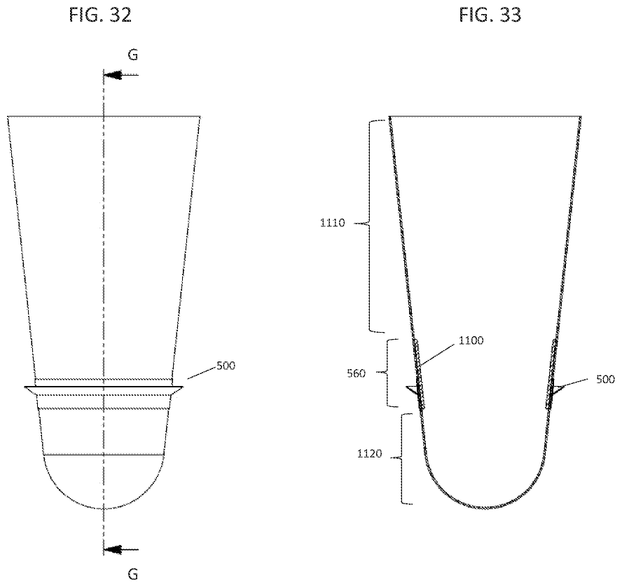

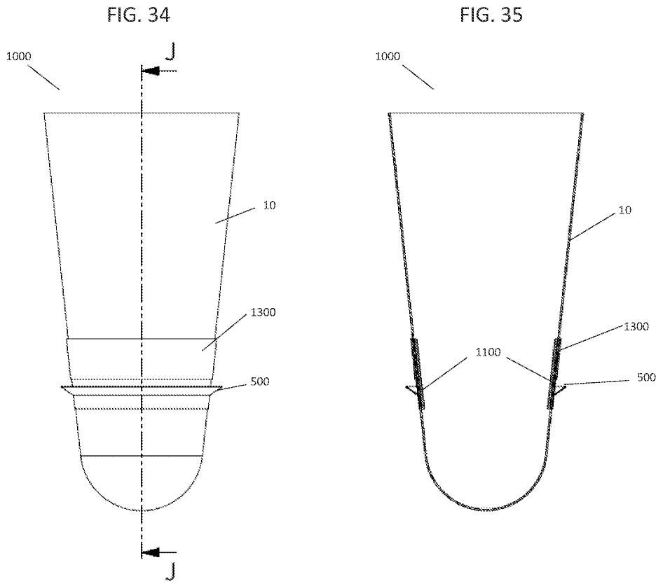

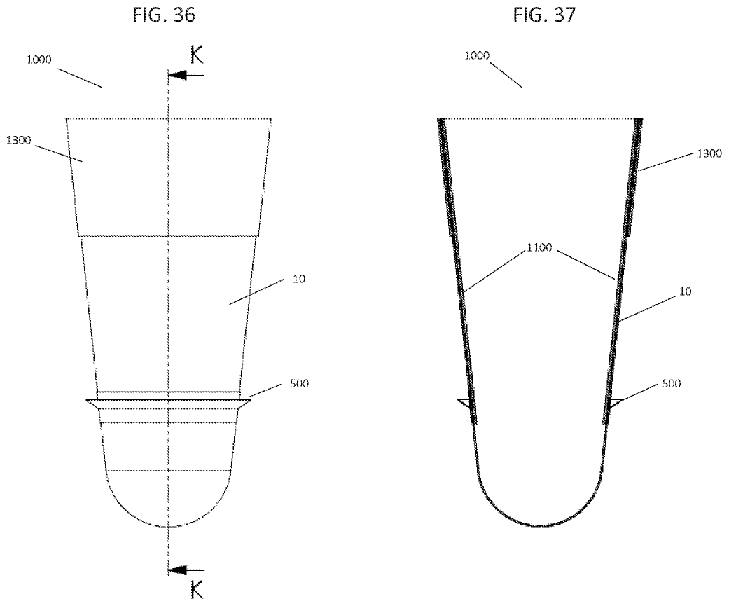

Alternative arrangements of the internal grip layer 1100 and external grip layer 1300 are shown in FIGS. 32-37. In FIGS. 32 and 33, the internal grip layer 1100 is only present in the intermediate region 560, leaving the proximal area 1110 and distal area 1120 free of any gripping material thereby allowing for additional breathability; this embodiment features no external grip layer 1300. In FIGS. 34 and 35, the internal grip layer 1100 is positioned similarly as in FIGS. 32 and 33 and additionally includes external grip layer 1300 positioned directly above the external sealing element 500 to provide additional vacuum when in use. Finally, FIGS. 36 and 37 have the internal grip layer 1100 as used in FIGS. 26 and 27 and an external grip layer 1300 as used in FIGS. 30 and 31.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. A suspension interface comprising:
a tubular stretchable fabric layer comprising a breathable fiber having an open proximal end and closed distal end;
at least one sealing element bonded to the tubular stretchable fabric layer at an intermediate region and comprising a sealing element base and a single engaging member which extends radially outward; and
an internal grip layer located on an inner face of the tubular stretchable fabric layer further comprising an impermeable elastomeric material.

2. The suspension interface of claim 1 wherein the synthetic fiber is nylon.

3. The suspension interface of claim 1 wherein the synthetic fiber is polyester.

4. The suspension interface of claim 1 wherein the elastomeric material is silicone.

5. The suspension interface of claim 1 wherein the elastomeric material is a thermoplastic elastomer comprising styrene blocks.

6. The suspension interface of claim 1 wherein the elastomeric material is urethane.

7. The suspension interface of claim 1 wherein the internal grip layer has a thickness between 1/64 inch and 1 inch.

8. The suspension interface of claim 1 wherein the tubular stretchable fabric layer further comprises an elastic yarn at the closed distal end to conform to a residual limb.

9. A suspension interface comprising:
a tubular stretchable fabric layer comprising a breathable fiber having an open proximal end and closed distal end;
at least one sealing element bonded to the tubular stretchable fabric layer at an intermediate region and comprising a sealing element base and a single engaging member which extends radially outward; and
an external grip layer located on an exterior face of the tubular stretchable fabric layer above the sealing element and further comprising silicone.

10. The suspension interface of claim 10 wherein the synthetic fiber is nylon.

11. The suspension interface of claim 10 wherein the synthetic fiber is polyester.

12. The suspension interface of claim 10 wherein the external grip layer has a thickness between 1/64 inch and 1 inch.

13. The suspension interface of claim 10 wherein the tubular stretchable fabric layer further comprises an elastic yarn at the closed distal end to conform to a residual limb.

14. A suspension interface comprising:
a tubular stretchable fabric layer comprising a breathable synthetic fiber having an open proximal end and closed distal end;
at least one sealing element bonded to the tubular stretchable fabric layer at an intermediate region and comprising a sealing element base and a single engaging member which extends radially outward;
an internal grip layer located on an inner face of the tubular stretchable fabric layer and comprising silicone; and
an external grip layer located on an exterior face of the tubular stretchable fabric layer and comprising silicone.

15. The suspension interface of claim 14 wherein the synthetic fiber is nylon.

16. The suspension interface of claim 14 wherein the synthetic fiber is polyester.

17. The suspension interface of claim 14 wherein the internal grip layer has a thickness between 1/64 inch and 1 inch.

18. The suspension interface of claim 14 wherein the external grip layer has a thickness between 1/64 inch and 1 inch.

19. The suspension interface of claim 14 wherein the tubular stretchable fabric layer further comprises an elastic yarn at the closed distal end to conform to a residual limb.
